(19) United States
(12) Patent Application Publication
(10) Pub. No.: US 2006/0251711 A1
(43) Pub. Date: Nov. 9, 2006

(54) Sterically stabilized carrier for aerosol therapeutics, compositions and methods for treating diseases of the respiratory tract of a mammal

(75) Inventors: Kameswari S. Konduri, Brookfield, WI (US); Sandhya Nandedkar, Brookfield, WI (US); Nejat Duzgunes, Mill Valley, CA (US); Pattisapu Ram Jogi Gangadharma, Irving, TX (US); Ramakrishna Pattisapu, Irving, TX (US)

Correspondence Address: F. LINDSEY SCOTT;LAW OFFICE OF F. LINDSEY SCOTT, 2329 COIT ROAD, SUITE B, PLANO, TX, 75075-3796, US

(73) Assignee: VGSK Technologies, Inc.

(21) Appl. No.: 11/442,907

(22) Filed: May 30, 2006

Related U.S. Application Data
(63) Continuation of application No. 10/769,034, filed on Jan. 30, 2004.
(60) Provisional application No. 60/498,609, filed on Aug. 28, 2003.
(60) Provisional application No. 60/498,546, filed on Aug. 28, 2003.

(51) Int. Cl. A61K 31/7048; A61K 31/7034; A61K 31/573; A61K 31/496; A61K 9/127; A61K 31/522; A61K 31/55; A61K 31/473

(52) U.S. Cl. 424/450; 514/35; 514/37; 514/39; 514/263.34; 514/253.08; 514/28; 514/29; 514/355; 514/217.05; 514/291; 514/171; 514/290; 514/649

(57) ABSTRACT

This invention relates to a sterically stabilized liposome carrier effective for the delivery of treatment to a mammal in a composition comprising a sterically stabilized liposome and a drug effective for the treatment of a mammal with the composition as an aerosol with the composition providing effective treatment for a period of time at least 1.5 times as long as the effective time for aerosol treatment with the drug alone. This invention also relates to a composition comprising the sterically stabilized liposome and the drug, as well as a method for treating a mammal for respiratory tract and lung disorders.

### RELATED CASES

This application is entitled to and hereby claims the benefit of the filing dates of U.S. Provisional Nos. 60/498,609 and 60/498,546, both filed Aug. 28, 2003.

### FIELD OF THE INVENTION

This invention is directed to a sterically stabilized liposome carrier effective for the aerosol delivery of a drug effectual in the treatment of the respiratory tract of a mammal and to a composition comprising a sterically stabilized liposome and a drug effective for the treatment of a mammal as an aerosol. The composition provides effective treatment for a period of time at least 1.5 times as long as the effective time for aerosol treatment of the mammal with a comparable quantity of the drug alone. A composition comprising the sterically stabilized liposome and the drug is disclosed as for use as an aerosol for the treatment of the respiratory tract of a mammal.

### BACKGROUND OF THE INVENTION

Asthma is a common disease that causes recurrent symptoms, repeated hospitalizations and an increased risk of sudden death. It is the most common childhood illness and affects five to ten percent of the population in North America. Asthma also accounts for the most hospitalizations of pediatric age people, the most missed school days and the most missed workdays at an estimated cost of $6.2 billion in 1988.

Asthma is characterized by acute bronchial restriction, chronic lung inflammation and airway hypersensitivity which results in chronic inflammation and airway remodeling that leads to progressive and possibly irreversible airway damage. The most effective therapy focuses on the early stages of the disease before the vicious cycle of inflammatory changes can become irreparable. The disease usually starts in early childhood and most commonly before five years of age. Thus, appropriate management of asthma in childhood may have a greater impact on the course of the disease than interventions later in life.

The mainstay of asthma treatment therapy is the use of anti-inflammatory drugs (i.e., inhaled corticosteroids). As a first line therapy for patients above five years of age, inhaled corticosteroids are usually given via a metered dose inhaler twice a day. Patients under five years of age are frequently given chromoline sodium three to four times a day via a nebulizer. A nebulizer form of Budesonide (BUD), which is a potent inhaled corticosteroid, given twice a day is being used as first line therapy in patients under five years of age in Europe and in Canada. It is now available in the United States.

Although current inhaled corticosteroids are very effective in preventing the massive inflammation that occurs with asthma, they do have some major drawbacks. One is that these drugs must be given at least daily to be effective. This daily dosage requirement may lead to non-adherence by the patient. Since adherence to daily use of inhaled corticosteriods by the patient is critical in interrupting the chronic inflammation that occurs in asthma, this becomes a focal issue for effective therapy. Further the effective use of a metered dose inhaler is very technique-dependent. Typically only three to eight percent of a given dose is delivered to the lungs using a metered dose inhaler. Additionally the inhaled corticosteroids have a short half-life in the body and have potential toxicity when used in higher doses. These are serious disadvantages to the use of corticosteroid drugs in conventional therapy. p In an abstract published by the present inventors in the Journal of Allergy Clinical Immunology entitled “Efficacy of Liposome Encapsulated Budesonide in Experimental Asthma,” February, 2001, Vol. 107, No. 2, it is disclosed that BUD encapsulated in sterically stabilized liposomes prevents asthma inflammation in lower doses given at less frequent intervals. Test results are summarized demonstrating an improvement. The abstract does not disclose a suitable sterically stabilized liposome, suitable types of sterically stabilized liposomes or any method for producing a suitable sterically stabilized liposome, for producing BUD encapsulated in a suitable sterically stabilized liposome or an administrative method for administering the sterically stabilized liposome containing BUD.

In view of the likelihood of detrimental effects based upon the use of the corticosteroids and the frequency with which the corticosteroids and other drugs are required, a continued effort has been directed to the development of improved techniques for administering a drug to the respiratory tract of a mammal so that it may be administered more effectively and so that the effectiveness of the drug can be achieved using smaller doses.

### SUMMARY OF THE INVENTION

The present invention comprises a sterically stabilized liposome carrier for combination with a drug, the sterically stabilized liposome being compatible with the respiratory tract of a mammal and effective to extend the effective life of the drug in the respiratory tract by a time equal to at least twice the effective life of the drug alone. The sterically stabilized liposome is adapted for aerosol administration to position the sterically stabilized liposome and the drug in the respiratory tract of a mammal.

The invention further comprises- a composition comprising a sterically stabilized liposome carrier in combination with a drug, the composition being adapted for aerosol administration, compatible with the respiratory tract of a mammal and effective to extend the effective life of the drug composition in the respiratory tract by a time equal to at least twice the effective life of the drug composition alone.

The invention further comprises a method for treating the respiratory tract of a mammal by administering an effective amount of a composition as an aerosol comprising a sterically stabilized liposome carrier in combination with a drug, the sterically stabilized liposome being compatible with the respiratory tract of a mammal and effective to extend the effective life of the drug in the respiratory tract of the mammal by a time equal to at least twice the effective life of the drug alone.

### BRIEF DESCRIPTION OF THE DRAWINGS

FIG. 1 is a graphical presentation of the histopathology scores for the mice groups tested in Example 1;

FIG. 2 is a graphical presentation of the eosinophil peroxidase (EPO) activity for the mice groups tested in Example 1;

FIG. 3 is a graphical presentation of the peripheral blood (PB) eosinophils for the mice groups tested in Example 1;

FIG. 4 is a graphical presentation of the serum IgE levels for the mice groups tested in Example 1;

FIG. 5 is a graphical presentation of the histopathology scores for the mice groups tested in Example 2;

FIG. 6 is a graphical presentation of the eosinophil peroxidase (EPO) activity for the mice groups tested in Example 2; and,

FIG. 7 is a graphical presentation of the airway hyperreactivity to methacholine (Mch) data for the mice groups tested in Example 2.

### DESCRIPTION OF PREFERRED EMBODIMENTS

Liposomes are well known materials that constitute primarily phospholipid bilayer vesicles of many types that can encapsulate a variety of drugs and are avidly phagocytosed by macrophages in the body. The various interactions of the liposomes can be generalized into four categories: (1) exchange of materials, primarily lipids and proteins with cell membranes; (2) absorption or binding of liposomes to cells; (3) cell internalization of liposomes by endocytosis or phagocytosis once bound to the cell; and, (4) fusion of bound liposomes with the cell membrane. In all these interactions, there is a strong dependence on lipid composition, type of cell, presence of specific receptors and many other parameters.

Liposomes have been used to provide drugs in mammal bodies, particularly when it is desired to apply the drugs to specific areas for specific applications. Liposomes have been used to encapsulate antibiotics, antiviral agents and the like and have been shown to enable enhanced efficacy against a variety of infectious diseases. A major drawback of liposomes is that they have a relatively short life in a mammal body. Most applications have used liposomes in the bloodstream.

To extend the life of liposomes in a mammal body, attempts have been made to develop sterically stabilized liposomes, which have a longer life in a mammal body. Attempts to extend the life of liposomes have included the use of poly(ethylene glycol), natural glycolipids, surfactants, polyvinyl alcohol, polylactic acid, polyglycolic acid, polyvinyl pyrrolidene, polyacrylamine and other materials in various combinations with the liposomes in attempts to provide sterically stabilized liposomes, which are effective for drug delivery and which are compatible with a mammal circulatory system. A wide variety of such sterically stabilized liposomes have been developed for a wide variety of drug deliveries for a wide variety of specific mammal disorders. The most prominent sterically stabilized liposomes utilize distearoylphosphatidylcholine as the primary phospholipid.

For use in the present invention, it has been necessary to produce sterically stabilized liposomes which are compatible with a mammal respiratory system and lungs, adapted for aerosol administration to the mammal and which have an extended life in the lungs and respiratory tract. For instance, the most commonly used sterically stabilized liposome uses distearoylphosphatidylcholine as the primary phospholipid. Due to its very high phase transition temperature, this lipid is not considered compatible with lung surfactant, which may contain dipalmitoyl lipids with shorter acyl chains and a lower phase transition temperature.

The sterically stabilized liposomes of the present invention have a composition such that they are readily administered to the mammal as an aerosol and will remain stable in the presence of serum and in the extra-cellular environment. They preferentially localize to the lungs, especially to areas of inflammation as commonly seen in asthma, i.e., in lung inflammation and in the airway hypersensitivity response. These sterically stabilized liposomes are amenable to nebulization. The combination of these sterically stabilized liposomes with drugs useful in the treatment of mammalian respiratory tract diseases has been shown herein with corticosteroids for the treatment of lung inflammation and airway hyper-responsiveness.

It is anticipated that these sterically stabilized liposomes will also be effective for the delivery of a wide variety of drugs for the treatment of respiratory and lung diseases. The effect of the sterically stabilized liposomes in combination with the encapsulated drug is more pronounced than currently available drug therapies. As demonstrated more thoroughly in the following examples, this stability may allow a drug, such as a corticosteroid, to be administered only once every one to two weeks. The dosage used in these treatments is typically the same or similar to that used on a daily basis. The effective life of the drug in the respiratory tract has thus been extended up to at least seven times the effective time of the drug alone. Sustained action of the drug has been obtained at much lower dosages with a reduction in toxicity risk and in cost. No suggestion in the prior art is known that extended life could be obtained with these sterically stabilized liposomes for aerosol drug treatments for asthma, particularly for lung inflammation and airway hyper-responsiveness using sterically stabilized liposomes adapted for use in the lungs and airway with drugs such as corticosteroids.

The sterically sterilized liposome carriers of the present invention, which are adapted for combination with a variety of drugs for use in the aerosol treatment of a respiratory tract in a mammal, comprise sterically stabilized liposomes that are compatible with the respiratory tract of a mammal and which are effective to extend the effective life of the drug in the respiratory tract by a time equal to at least twice the effective life of the drug alone. The sterically stabilized liposomes of the present invention are tailored to be compatible with naturally occurring fluids found in the lungs. The surfactants are also tailored to accommodate the surfactant nature of some of the fluids found in the lungs so that the sterically stabilized liposomes of the present invention provide long stability in the lungs and when used to encapsulate or combine with selected drugs have been found to be effective to extend the effective life of drugs administered using the sterically stabilized liposome carriers of the present invention.

The sterically stabilized liposomes of the present invention comprise phosphatidylcholine. These materials may be synthetically derived or they may be derived from chicken eggs or soybeans. If derived from eggs they contain acyl groups having varying numbers of carbon atoms, dependent upon the variety and diet of the chicken that produces the eggs. The phosphatidylcholine is typically present in a relatively significant quantity in the sterically stabilized liposomes and may comprise the only head group for the sterically stabilized liposomes.

Alternatively, the sterically stabilized liposomes may also include significant quantities, up to 50%, of head groups comprising phosphatidylglycerol. This mixed material is considered to be somewhat more compatible with lung fluids than is phosphatidylcholine alone.

A further component of the sterically stabilized liposomes may be poly(ethylene glycol), in the molecular range from about 500 to about 5,000 daltons.

Any of the head groups or the poly(ethylene glycol), may be attached to acyl groups containing from about 8 to about 18 carbon atoms. Preferably, from about 8 to about 18 carbon atoms are present in the acyl groups. Such groups comprise distearoyl, stearoyl oleoyl, stearoyl palmitoyl, dipalmitoyl, dioleoyl, palmitoyl oleoyl and dipalmitoleoyl.

If shorter chains are used, such as palmitoyl, dimyristoyl, didodecanoyl, didecanoyl or dioctanoyl, the poly(ethylene glycol)-lipid is likely to exchange into biological milieu. This may in some instances permit the liposome to better partition onto lung surfactant after shedding or exchanging its poly(ethylene glycol) moiety.

Desirably, the sterically stabilized liposomes may be tailored to the particular mammalian lung system contemplated. It is considered, however, that such sterically stabilized liposomes will fall within the criteria defined above for the liposomes.

Further the sterically stabilized liposomes may comprise at least one of phosphatidylcholine, phosphatidylglycerol, and poly(ethylene glycol)distearyolphosphatidyidiethanolamine, lipid conjugated polyoxyethylene, lipid conjugated polysorbate, or lipids conjugated to other hydrophilic steric coating molecules safe for in vivo use.

Particularly preferred material is phosphatidylcholine, phosphatidylglycerol, poly(ethylene glycol)-distearyolphosphatidyidiethanolamine. This sterically stabilized liposome was used in the tests shown in Examples 1 and 2.

The drugs, which can be combined with the sterically stabilized liposomes of the present invention, comprise substantially any drug that is useful against diseases of the respiratory tract of a mammal. It is anticipated that most drugs that are useful in such treatments will be compatible with the sterically stabilized liposomes. Typically, the combination of the sterically stabilized liposomes and the drugs are administered via an aerosol to the respiratory tract.

Types of drugs that can be included in the sterically stabilized liposome are not limited so long as the formation and stability of the sterically stabilized liposome is not adversely affected.

The combined sterically stabilized liposomes and drugs form unilamellar or multilamellar vesicles of sizes from about 0.05 to about 10 micrometers. Preferably the composition is prepared to have substantially homogeneous sizes in a selected size range, with the average diameter typically from about 0.05 to about 0.8 micrometers. One method for obtaining the desired size is extrusion of the composition through polycarbonate membranes having pores of a selected size, such as from about 0.05 to about 2 micrometers.

Some drugs that are considered particularly suitable are inhaled corticosteroids; such as, Budesonide, Flunisolide, Triamcinolone, Beclomethasone, Fluticasone, Mometasone, Dexamethasone, Hydrocortisone, Methylprednisolone, Prednisone, Cotisone, Betamethasone, or the like. Some other suitable drugs are bronchodilators; such as Terbutaline, Albuterol, Ipratropium, Pirbuterol, Epinephrine, Salmeterol, Levalbuterol, Formoterol, or the like.

Other drugs that are also considered to be suitably administered using the sterically stabilized liposomes of the present invention include, but are not limited to, Leukotriene inhibitors; such as Montelukast, Zafirlukast, Zileuton, or the like, can also be used, as well as antihistamines; such as Loratadine, Cetirizine or the like, Anti-Tuberculosis drugs for M TB or atypical mycobacteria; such as, Isoniazid, Ethambutol, Pyrazinamide, Rifamycin; Rifampin, Streptomycin, Clarithromycin, or the like can also be suitable. Other drugs; such as the Serine lung protease inhibitors Azelastine, and Theophylline; and other peptides, such as those that relate to Allergy Immunotherapy for indoor and outdoor allergens, or the like, may also be considered suitable. Additionally, amikacin, gentamicin, tobramicin, rifabutin, rifapentine, sparfloxacin, ciprofloxacin, quinolones, azithromycin, erythromycin, isoniazid, or the like, can be considered to be useful.

Most previously disclosed sterically stabilized liposomes have been used in attempts to extend the effective life of drugs used in the bloodstream of mammals. These sterically stabilized liposomes must exist in a radically different environment than in the respiratory tract of a mammal. Particularly in the lungs, certain surfactant requirements exist for materials that are compatible with the fluids in the lungs and the like. Further the sterically stabilized liposomes delivered to the lungs are not as susceptible to attack by phagocytic cells as are sterically stabilized liposomes used to position drugs in the bloodstream, which are eventually cleared mostly by liver and spleen macrophages. Further most uses of sterically stabilized liposomes in combination with drugs in the bloodstream are administered via intravenous injections. While it is not clear what mechanisms exist that permit sterically stabilized liposomes to exist for longer periods of time in certain portions of the body than would be anticipated for liposomes that were not sterically stabilized, it is clear that the sterically stabilized liposomes of the present invention are remarkably stable in the respiratory tract environment and are effective to greatly extend the effective life of drugs used to treat various ailments of the respiratory tract.

The preparation of the sterically stabilized liposomes, the combination of the corticosteroid drug with the sterically stabilized liposomes, and treatments of mice according to the present invention, are demonstrated in the following examples.

### EXAMPLE 1

Methods

Animals

Six week-old male C57 black 6 mice were purchased from Charles River Laboratories, Inc., Willmington, Mass. The animals were provided with an ovalbumin-free diet and water ad libitum and were housed in an environment-controlled, pathogen-free animal facility. All animal protocols were approved by the Animal Care Committee of the University of Illinois at Chicago, the Medical College of Wisconsin and the Zablocki Veterans Administration Medical Center, and were in agreement with the National Institute of Health's guidelines for the care and use of laboratory animals.

Ovalbumin Sensitization

The animals were sensitized with ovalbumin (OVA). On day 0, each mouse was anesthetized with methoxyflurane given by inhalation. A fragmented heat-coagulated OVA implant was inserted subcutaneously on the dorsal aspect of the cervical region.

For a ten-day period (days 14-24), each mouse was given a 30-minute aerosolization of a 6% OVA solution on alternate days. This method of sensitization led to significant elevations in eosinophil peroxidase (EPO), peripheral blood (PB) eosinophils, and serum IgE levels, along with lung inflammation as seen on histophathologist by day 24.

Treatment Groups

Therapy was initiated on day 25, the day after the OVA sensitization was complete. Sensitized animals received nebulized treatments for four weeks as follows:
a) BUD (20 μg) encapsulated in sterically stabilized liposomes, administered once a week (Wk-S-BUD group);
b) BUD (20 μg) without liposome encapsulation, administered daily (standard therapy—Daily BUD group);
c) BUD (20 μg) encapsulated in conventional liposomes, administered once a week (Wk-C-BUD group);
d) buffer-loaded (empty) sterically stabilized liposomes, administered once a week (Wk-Empty-S group); and,
e) BUD (20 μg) without liposome encapsulation, administered once a week (Wk-BUD group).

Each of the nebulization doses was given at a volume of 1 ml. for 2 minutes through use of a chamber in which the mouse was allowed to breathe freely. All treatment groups were compared with either sensitized untreated or unsensitized (Normal) mice.

The amounts of lipid used for the Wk-Empty-S group were based on the amount of lipid nebulized for each of the BUD-encapsulated liposomes (1.39 μmol for the sterically stabilized liposomes and 3.19 μmol for the conventional liposomes).

The dose of BUD chosen was based on preliminary dose-response studies with 5 to 50 μg of BUD as follows.

Each day, 5, 10, 15, 20 or 50 μg of BUD was administered via nebulization to groups of sensitized mice, and the dose-dependent effects on the inflammatory parameters were evaluated. These data were compared with data for either a group of sensitized untreated mice (Sens group) or a group of unsensitized mice (Normal group). A 20 μg dose of BUD was shown on histopathologic examination to effectively decrease EPO activity in bronchoalevolar lavage fluid (BAL), PB eosinophils and inflammation of the lung tissues, along with other inflammatory parameters, without evidence of toxicity to the spleen, liver, bone.morrow or gastrointestinal tract. In addition, there were no granulomas or abnormalities in any of the tissues evaluated.

Each study group consisted of 20 mice and was followed for a four-week period. Five animals from each treatment group and from each of the two control groups, sensitized and unsensitized, were euthanized by means of an overdose of methoxyflurane inhaled 24 hours after the first treatments were given and then at weekly intervals for four weeks. At each time point, measurements of EPO in BAL, PB eosinophils, and total serum IgE levels were obtained and histopathologic examination of the lung tissues was performed.

Drugs and Reagents

BUD for daily therapy was diluted from premixed vials (0.25 mg/ml) commercially available from Astra Pharmaceutical, Wayne, Pa., and was administered via a Salter Aire Plus Compressor, Salter Labs, Irvine, Calif. BUD for encapsulation and N-2-hydroxethylpiperzine-N′-2-ethanesulfonic acid (HEPES) was purchased from Sigma Chemical, St. Louis, Mo. Phosphatidylcholine (PC), phosphatidylglycerol (PB), and poly(ethylene glycol)-distearoylphosphatidylethanolamine (PEG-PE) were obtained from Avanti Polar Lipids, Alabaster, Ala. Cholesterol was purchased from Calbiochem, La. Jolla, Calif. NaCl and KCl were purchased from Fisher Scientific, Pittsburgh, Pa.

Liposome Preparation

BUD was encapsulated into either sterically stabilized (phosphotidylglycerol-phosphotidylcholine-poly(ethylene glycol)distearoylphosphatidylethanolamine-cholesterol) or conventional (phosphotidyl-glycerolphosphotidylcholine-cholesterol) liposomes through use of a protocol derived from the protocol described by Gangadharam, et al. A portion of the cholesterol used in control liposomes was replaced by BUD (dissolved in chloroform-methanol-2:1) during the preparation of the lipid mixture. Lipids were dried onto the sides of a round-bottomed glass flask or glass tube by rotary evaporation. The dried film was then hydrated by adding sterile 0140 mmol/L, NaCl and 10 mmol/L HEPES (pH 7.4) and vortexing. The resulting multilamellar liposome preparations were extruded 21 times through polycarbonate membranes (either 0.2 or 0.8 μm in pore diameter), (Nuclepore, Pleasanton, Calif.) through use of an Avestin extrusion apparatus, Toronto, Canada.

Histopathology Observations

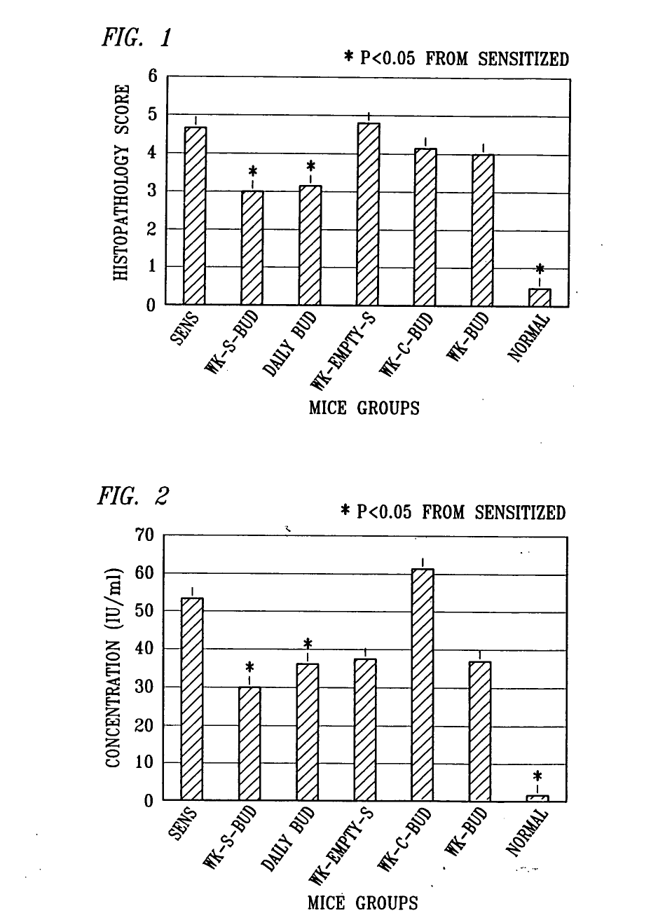

Histopathologic examination was performed on lungs that were removed and fixed with 10% phosphate-buffered formalin. Tissue samples were taken from the trachea, bronchi, large and small bronchioles, interstitium, alveoli and pulmonary blood vessels. The tissues were embedded in paraffin, sectioned at a thickness of 5 μm, stained with hematoxylin and eosin and analyzed through use of light microscopy at a magnification of ×100. Coded slides were examined by a veterinary pathologist, in a blinded fashion, for evidence of inflammatory changes, including (1) bronchiolar epithelial hyperplasia and wall thickening, (2) bronchiolar, peribronchiolar, and perivascular edema and (3) accumulation of eosinophils, neutrophils and mononuclear inflammatory cells. Each of the parameters evaluated was given an individual number score. The cumulative score was obtained through use of the individual scores; inflammation was designated as none (score 0), mild (score 1-2), moderate inflammation (score 3-4) or severe inflammation (score 5-6). The histopathology score for the Groups are shown in FIG. 1.

Eosinophil Peroxidase (EPO) Activity in Bronchoalveolar Lavage (BAL) Fluid and Peripheral Blood (PB) Eosinophils

When each mouse was euthanized, the trachea was exposed and cannulated with a ball-tipped 24-gauge needle. The lungs were lavaged three times with 1 ml PBS. All of the washings were pooled and the samples were frozen at −70° C. The samples were later thawed and assayed for determining EPO activity.

EPO in the BAL was assessed as follows. A substrate solution consisting of 0.1 mol/L sodium citrate, 0-phenylenediamine, and H₂O₂ (3%), pH 4.5 was mixed with BAL supernatants at a ratio of 1:1. The reaction mixture was incubated at 37° C. and the reaction was stopped by the addition of 4 N H₂SO₄. Horseradish peroxidase was used as a standard EPO activity (in international units per milliliter) was measured by spectrophotometric analysis at 490 nm. The percentages of eosinophils were obtained by counting the number of eosinophils in 100 white blood cells under a high-power field scope (×100) from the PB smears stained with Wright-Giemsa stain.

Total Serum IgE

Ninety-six well flat bottom plates (Fisher Scientific) were coated with 100 μL per well of 2 μg/ml rat antimouse IgE monoclonal antibody (BD, PharMingen, San Diego, Calif.), and incubated overnight at 4° C. Serum was added at a dilution of 1:50 and incubated overnight at 4° C. Purified mouse IgE (k isotype, small b allo-type anti-TNP:BD PharMingen) was used as the standard for total IgE. The samples were incubated for one hour with biotin-conjugated rate antimouse IgE (detection antibody purchased from Southern Biotechnology, Birmingham, Ala.).

Data Analysis

Data analysis was performed using the Student t test. P values of less than 0.05 were considered significant. Statistical analysis was performed through use of weekly serial measurements from each group. Cumulative data for the four-week period for each study group are presented.

Results

Over the four-week period, there were no significant increases or decreases in inflammation within each group according to weekly measurements for all of the inflammatory parameters being evaluated.

Histopathology

Significant reduction in total lung histopathologic score (FIG. 1) was noted with weekly treatments of BUD encapsulated in sterically stabilized liposomes in comparison with what was seen in the sensitized untreated mice and this reduction was similar to that seen with the daily BUD therapy. Similar decreases were not observed. with the weekly BUD encapsulated in conventional liposome treatment, the weekly BUD treatment or the weekly empty sterically stabilized liposomes treatment.

There was also a significant decrease in lung inflammation in the Wk-S-BUD group in comparison with the Wk-Empty-S group and the Wk-BUD group. There was no significant difference between the Daily BUD group and the Wk-S-BUD group.

The lung tissues from the sensitized untreated mice had persistent and significant inflammation, including accumulation of inflammatory cells with considerable numbers of eosinophils in bronchiolar, peribronchiolar and perivascular tissues, along with significant submucosal thickening and epithelial hyperplasia, during the four-week period.

EPO Activity

Weekly treatments with BUD encapsulated in sterically stabilized liposomes significantly decreased the EPO activity in the BAL in comparison with what was seen in the sensitized untreated mice and they were comparable to daily BUD therapy. The Wk-BUD, the Wk-Empty-S and the Wk-C-BUD groups did not show any significant decreases in EPO activity. These test results are shown in FIG. 2.

PB Eosinophils

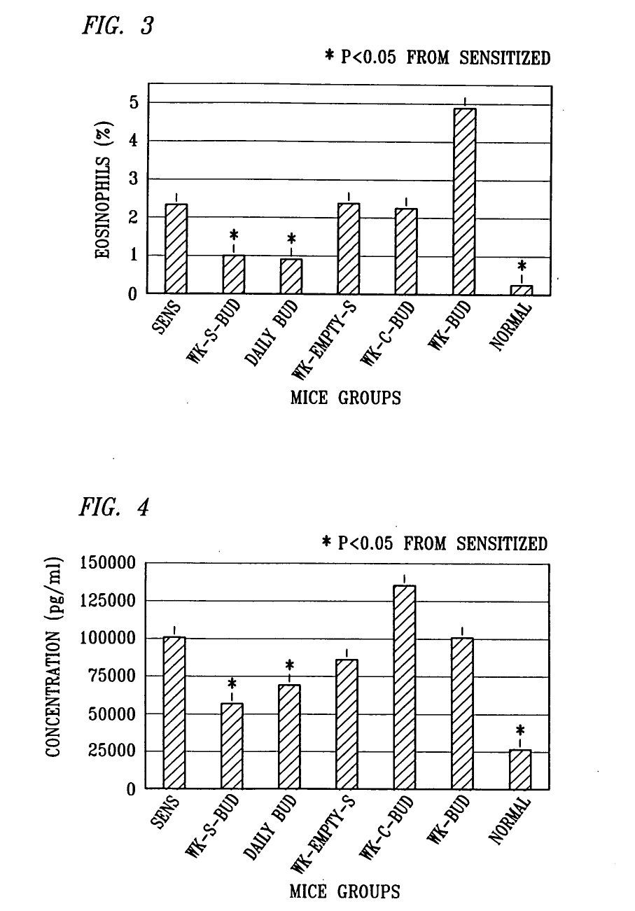

Therapy with weekly BUD encapsulated in sterically stabilized liposomes and therapy with daily BUD significantly decreased PB eosinophils in comparison with what was seen in Sens group. None of the other treatment groups, including the Wk-C-BUD group, showed significantly decreased PB eosinophils in comparison with the Sens group. These test results are shown in FIG. 3.

Total Serum IgE

Treatment with weekly BUD encapsulated in sterically stabilized liposomes and treatment with daily BUD significantly lowered the total serum IgE level. The total serum IgE level was not significantly reduced in the Wk-C-BUD group or any of the other treatment groups in comparison with the Sens group. These test results are shown in FIG. 4.

In the present study, it was demonstrated that BUD encapsulated in sterically stabilized liposomes, given once a week, reduced inflammation as effectively as the same dosage of BUD given once a day. Weekly treatments with free BUD, BUD encapsulated in conventional liposomes and empty sterically stabilized liposomes did not have comparable effects.

Inhaled corticosteroids are the most commonly prescribed anti-inflammatory drugs in asthma therapy. However, the need for daily dosing might lead to problems of noncompliance and treatment failures, which might result in increased hospitalizations and complications. This is the first study to investigate weekly therapy with BUD encapsulated in sterically stabilized liposomes to treat experimental asthma. The results show that sterically stabilized liposomes have a unique capacity to delivery BUD effectively to mammalian lungs, requiring only a fraction of the dosage and a less frequent dosing interval in comparison with conventional therapy.

The most important aim of the present study was to determine whether use of this drug delivery system alters the significant inflammatory airway response of asthma. Levels of immunologic markers implicated in the progression of asthma such as EPO activity in BAL, PB eosinophils, serum IgE levels and lung inflammation as seen on histologic examination were decreased with the novel mode of drug delivery.

The finding of worsening inflammation in 2 of the groups WK-BUD and Wk-C-BUD was an unexpected finding. A possible mechanism is that Wk-BUD therapy or Wk-C-BUD therapy produces an initial rapid response followed by a rebound effect on inflammation. No previous reports on the effects of weekly therapy with BUD were known to the present inventors. In addition, it has been shown that steroids encapsulated in conventional liposomes diffuse rapidly from these sterically stabilized liposomes, it is thus possible that none of the BUD encapsulated in the conventional liposomes resulted in sustained delivery to the lungs.

The decrease in PB blood cosinophils seen in this study was consistent with previous reports that inhaled steroids reduce the production of pro-inflammatory cytokines.

BUD encapsulated in sterically stabilized liposomes has been shown to significantly decrease inflammation in experimental asthma. The animals tolerated the therapy without adverse side effects, such as abnormal weight gain, irritability, respiratory distress and histologic abnormalities in the bone marrow, bone, spleen, liver or gastrointestinal tract.

Encapsulation in sterically stabilized liposomes can thus be a safe and effective vehicle for delivery of inhaled steroids to asthmatic lungs. This unique drug delivery method, provides an alternative to daily BUD therapy, with the potential to reduce toxicity and improve compliance for inhaled steroid therapy in asthma.

### EXAMPLE 2

Methods

Further tests were run to demonstrate that BUD is effective in reducing airway hyper-responsiveness to metacholine. These tests demonstrate that the sensitivity of the ainvay that causes excessive coughing and the like in asthma sufferers is effectively treated by the use of BUD in a composition comprising BUD and the sterically stabilized liposomes.

These tests were run as follows.

Animals

Six week-old male C57Black 6 (C57/B16) and BALBc mice were purchased from Charles River Laboratories, Inc., Willmington, Mass. A/J mice were purchased from Jackson Laboratories, Bar Harbor, Me. The animals were provided an ovalbumin-free diet and water ad libitum and were housed in an environmentally controlled, pathogen-free animal facility. All animal protocols were approved by the Animal Care Committee of the Medical College of Wisconsin and the Zablocki Veterans Administration Medical Center, and were in agreement with the National Institutes of Health's guidelines for the care and use of laboratory animals.

Ovalbumin Sensitization

The animals were sensitized with ovalbumin (OVA) as described previously. Briefly, on day 0, a subcutaneous OVA implant was placed. On day fourteen and on alternate days, through day 24, the mice were given a 30-minute aerosolization of 6% OVA solution for a period of 10 days.

Comparison of C57/B16, A/J, and BALBc Mice

Using our method of ovalbumin-sensitization, C57/B16, A/J, and BALBc mice were compared in their AHR to Mch challenge, since previous studies have demonstrated an interstrain variability in AHR to Mch challenge. There was no significant strain difference in AHR to Mch challenge between the sensitized C57/B16 and A/J or BALBc mice. Therefore, this study was conducted with C57/B16 mice.

Treatment Groups

Drug therapy was given only to the sensitized C57/B16 mice. Therapy was initiated on day 25, one day after the OVA sensitization was completed. Sensitized (S) animals received nebulized treatments for four weeks of either: (1) 20 μg BUD encapsulated in sterically stabilized liposomes, once a week (L); (2) 20 μg BUD without liposomes encapsulation given daily (D); (3) buffer-loaded (empty) stealth liposomes, once a week (E); and (4) 20 μg BUD without liposome encapsulation, once a week (W). All treatment groups were compared to untreated, sensitized and unsensitized normal (N) C57/BV16 mice.

Each study group consisted of 20 C57/B16 mice and was followed for four weeks. The nebulization doses were all given at a volume of 1 ml for 2 minutes, using a chamber that allowed the mouse to breathe freely. The time periods between sensitization, treatment and pulmonary mechanics measurements were the same for all groups.

As previously described, the amounts of lipids used for the empty liposome control group were based on the amount of lipid nebulized for the BUD-encapsulated liposome (1.39 μmol) for the sterically stabilized liposomes). The 20 μg dose of BUD chosen was based on the results from our previous studies.

Drugs and Reagents

BUD for daily therapy was diluted from premixed vials containing 0.025 mg/ml and commercially available from Astra Pharmaceuticals, Wayne, Pa. The drug was administered via a Salter Air Plus Compressor, commercially available from Salter Labs, Irvine, Calif. BUD for encapsulation and N-2-hydroxethylpiperzine-N′-2-ethanesulfonic acid (HEPES) was purchased from Sigma Chemical Co., St. Louis, Mo.

Phosphatidylcholine, phosphatidylglycerol and poly(ethylene glycol), -distearoylphosphatidylethanolamine were obtained from Avanti Polar Lipids, Alabaster, Ala. Cholesterol was purchased from Calbiochem, La Jolla, Calif. NaCl and KCl were purchased from Fisher Scientific, Pittsburgh, Pa.

Liposome Preparation

BUD was encapsulated into the sterically stabilized liposomes (phosphatidylcholine, phosphatidylglycerol-poly(ethylene glycol)distearoylphosphatidylethanolamine-cholesterol) using a protocol as previously described. Briefly, a portion of the cholesterol used in control liposomes was replaced by BUD (dissolved in chloroform: methanol, 2:1) during the preparation of the lipid mixture. Lipids were dried onto the sides of a round-bottom glass flask or glass tube by rotary evaporation.

The dried film was then hydrated by adding sterile 140 mM NaCl, 10 mM at a HEPES pH 7.4 and vortexed. The resulting multilamellar liposome preparations were extruded 21 times through either 0.2 or 0.8 μm pore diameter polycarbonate membranes (Nuclepore, Pleasanton, Calif.) using an Avestin (Toronto, Canada) extrusion apparatus.

AHR to Mch Challenge

Pulmonary mechanics were studied as follows:

Pulmonary resistance measurements were made after four weeks of therapy. As an antigen challenge and to demonstrate sensitization, an aerosolized dose of 6% ovalbumin gas given to each animal 24 hours before the evaluation of the pulmonary mechanics.

The animals were anesthetized with an intraperitoneal injection of a solution of ketamine and xylazine (40 mg/kg body weight for each drug). A 20 mg/kg body weight maintenance dose of pentobarbital sodium was given before placement in the body plethysmogragh. The doses were titrated to maintain a steady level of anesthesia without causing significant respiratory depression.

A tracheotomy was performed and a tracheotomy tube was placed in each animal. A saline-filled polyethylene tube with side holes was placed in the esophagus and was connected to a pressure transducer for measurement of pleural pressure. The mice were then placed in a body plethysmograph chamber for measurements of flow, volume and pressure.

The tracheostomy tube was connected to a tube through the wall of the plethysmograph allowing the animal to breathe room air spontaneously. The esophageal catheter was connected to a pressure transducer. Proper placement of the esophageal catheter was verified using assessments of pressure-volume-flow loops. A screen pneumotachometer and a Valadyne differential pressure transducer were used to measure flow in and out of the plethysmograph.

The frequency response of the plehtysmograph-pneumotachometer system determined using the volume oscillator of an Electromechanical Multifunction Pressure Generator available from Millar Instruments, Inc., Houston, Tex., was such that the amplitude decreased by less than 10% to a frequency of 12 Hz. The maximum breathing frequency in the mice studied was 4.3 Hz.

Signals from the pressure transducer and the pneumotachometer were processed using a Grass polygraph (Model 7) recorder. The flow signal was integrated using a Grass polygraph integrator (Model 7P10) to measure corresponding changes in pulmonary volume. Pressure, flow and volume signal outputs were digitized and stored on computer using an analog-to-digital data acquisition system (CODAS—available from Dataq Instruments, Inc., Akron, Ohio). The pressure and volume signals were also displayed to verify catheter placement and monitor the animal during the experiment.

The digitalized data were analyzed for dynamic pulmonary compliance, pulmonary resistance, tidal volume, respiratory frequency and minute ventilation from about six to ten consecutive breaths at each recording event. Compliance and resistance were calculated from pleural pressure, airflow, and volume data.

To correct for the resistance of the tracheal cannula, the pressure-flow curve relationship fro the cannula alone was measured. It was found to have resistance of 0.3 cmH₂) mol⁻¹s, which was then subtracted from the total resistance, measured with the animal in place to determine the pulmonary resistance. Mch challenge was performed after baseline measurements were obtained. Mch (Sigma Chemicals, St. Louis, Mo.) was injected intraperitoneally at three-minute intervals in successive cumulative doses of 30, 100, 300, 1,000 and 3,000 μg.

Eosinophil Peroxidase Activity (EPO) in BAL

EPO activity in the BAL was measured in all the experimental groups with and without Mch challenge. At the time of sacrifice, the trachea was exposed and cannulated with a ball-tipped 24-gauge needle. The lungs were ravaged three times with 1 ml PBS. All washings were pooled and the samples were frozen at −70° C. The samples were later thawed and assayed to determine EP activity.

Histopathology Observations

Histopathological examinations were performed as follows:

The lungs were removed and fixed with 10% phosphate buffered formalin. Tissue samples were taken from the trachea, bronchi, large and small bronchioles, interstitium, alveoli, and pulmonary blood vessels. The tissues were embedded in paraffin, sectioned at 5 μm thickness and stained with hematoxylin and eosin and analyzed using light microscopy at 100× magnification.

Coded slides were examined by a veterinary pathologist in a blinded fashion for evidence of inflammatory changes, including bronchiolar epithelial hyperplasia and wall thickening, brorchiolar, peribronchiolar and perivascular edema and accumulation of eosinophils, neutrophils, and mononuclear inflammatory cells. Each of the parameters evaluated was given an individual number score. The cumulative score was obtained using the individual number scores and was designated as no inflammation (0), mild inflammation (1-2), moderate inflammation (34) and severe inflammation (5-6).

Data Analysis

Cumulative data from the four-week period from each study group are presented as mean±standard error of the mean (SEM). One-way ANOVA with Tukey-Kramer multiple comparison data analysis was used for Mch responses using SigmaStat Statistical Software (SPSS Science). EPO activity analysis was performed using the Student I test. A p<0.05 was considered to be statistically significant for all of the above statistical comparisons.

Results

AHR to Mch Challenge

The baseline airway resistance (RL) in normal mice before challenge with Mch was 1.14 cm H₂O) ml⁻¹s. The baseline RL was greater in the E and D groups. Five mice (1L, 1E, 2D and 1W) survived only up to the lmg dose of Mch. The R_{L} was significantly increased in these animals from 2 to 5 times compared to the mice in the respective group. For the subsequent data analysis, only data from mice for which a complete set of data was available were used for analysis.

At a cumulative dose of 1 mg Mch, R_{L} was increased in all groups. There was no significant difference between the airway responsiveness of any of the groups of sensitized mice receiving treatment compared to the untreated sensitized (S) mice. Only the sensitized animals treated with D had an airway response that was significantly greater than the normal unsensitized mice.

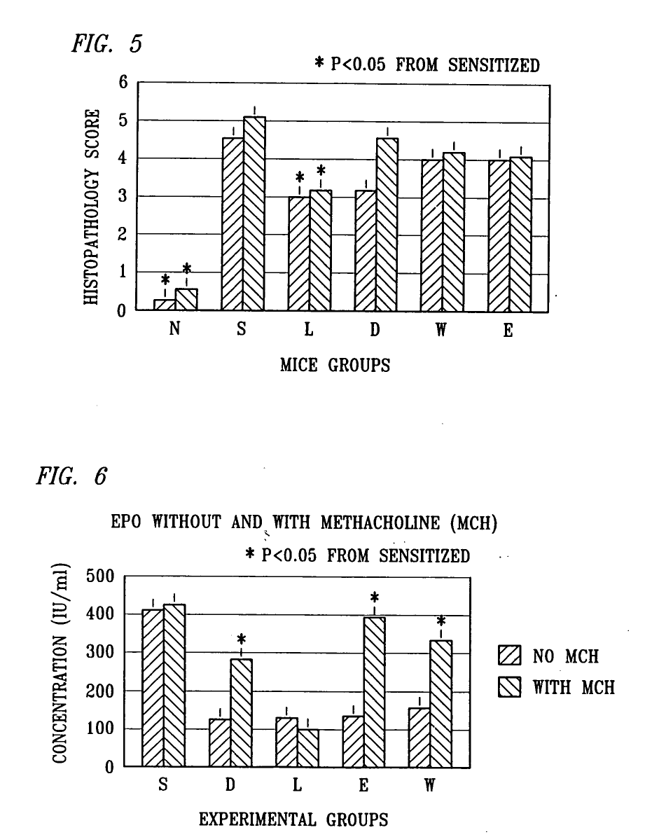

All the treatment groups except the L group given Mch at a cumulative dose of 3 mg demonstrated a significant increase in R_{L} compared to the normal unsensitized mice. There was no significant difference in R_{L} between the normal mice (N) and the L mice. These were the only two groups of mice with an R_{L} significantly less than the S mice. These test results are shown in FIG. 5.

Eosinophil Peroxidase Activity (EPO)

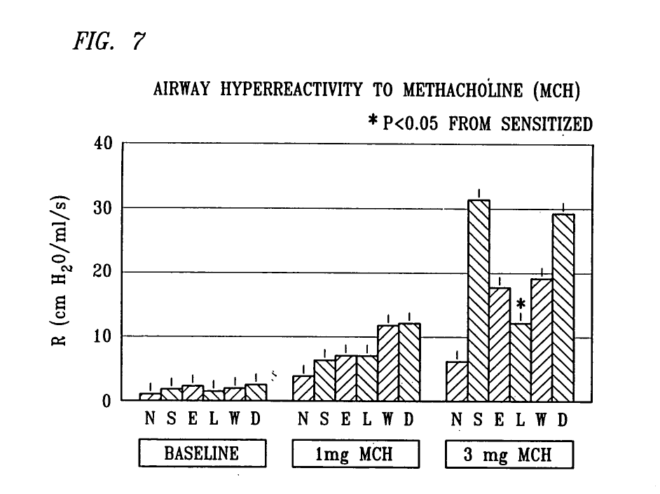

In the groups that did not undergo Mch challenge, the L (p<0.001) and the D (p<0.001) treatment groups significantly decreased the EPO activity when compared to the S group (FIG. 6). W (p<0.419) and the E (p<0.213) treatment groups did not show a significant decrease in EPO activity.

There was no significant difference in the EPO activity with or without Mch challenge in the L treatment group (p<0.68), whereas EPO activity of all other study groups was increased. This data is shown in FIG. 6.

Histopathology

There was a significant reduction in total lung histopathology score (FIG. 7) without Mch challenge, with L (p<0.030) and D (p<0.030) treatment groups when compared to the S group. Similar decreases were not observed with the other treatment groups.

With Mch challenge, only the L group had a significant decrease in total histopathology score (p<0.0009) when compared to the S group. None of the other treatment groups showed a similar reduction with Mch challenge.

In Example 2, it has been demonstrated that BUD encapsulated in sterically stabilized liposomes administered weekly by inhalation reduces the airway hyper-responsiveness as measured by the Mch challenge. In this study, the weekly treatment with the sterically stabilized liposomes and BUD was the only treatment group that had a significant decrease in airway hyper-responsiveness, EPO activity and pulmonary inflammation with Mch challenge. The daily or weekly treatments with unencapsulated BUD did not significantly decrease airway hyper-responsiveness, EPO activity or pulmonary inflammation with Mch challenge.

This study demonstrates that BUD encapsulated in sterically stabilized liposomes and administered weekly by inhalation reduces AHR as measured by Mch challenge. The experiments were conducted with ovalbumin-sensitized C57/B16 mice since no significant interstrain differences in AHR with sensitized C57/B16, A/J, or BALBc mice were found.

Group L was the only treatment group that had a significant decrease in AHR, EPO activity and pulmonary inflammation with Mch challenge. In contrast, daily or weekly treatments with unencapsulated BUD did not significantly decrease AHR, EPO activity or pulmonary inflammation with Mch challenge. The test results differed from previous studies that showed a decrease in AJR to Mch challenge with daily, unencapsulated BUD therapy. A possible explanation for the difference in these results is that in this study a lower dose of unencapsulated BUD was given once a day instead of twice a day. In addition, the size of the liposome was smaller than the daily BUD delivery vehicle.

Imaging studies and immunohistochemical studies of the asthmatic airways have demonstrated that distal airways have significant inflammation which is not adequately treated with inhaled steroids and contribute to AHR and remodeling of the lungs. The effectiveness of inhaled anti-inflammatory medications in decreasing pulmonary inflammation and AHR would possibly improve if more drug could be deposited in the small and large airways.

The encapsulation of BUD into sterically stabilized liposomes enables it to reach the distal airways more efficiently and effectively. In addition, sterically stabilized liposomes may have a surfactant-like effect that may allow for better penetration of the drug to the distal small airways and alveoli.

This is the first study to show the efficacy of BUD encapsulated in sterically stabilized liposomes as a treatment that can be administered once a week to decrease AlIR to Mcli challenge, comparable to normal mice. This new treatment modality provides a method for using very low doses and less frequent dosing intervals of BUD to decrease both pulmonary inflammation and AHR that is associated with asthma.

Similar tests have been done with triamcinolone, with similar results.

While the present invention has been described by reference to certain of its preferred embodiments, it is pointed out that the embodiments described are illustrative rather than limiting in nature and that many variations and modifications are possible within the scope of the present invention. Many such variations and modifications may be considered obvious and desirable by those skilled in the art based upon a review of the foregoing description of preferred embodiments.

What is claimed is:

1. A sterically stabilized liposome carrier for combination with a drug for aerosol administration, the sterically stabilized liposome being compatible with a respiratory tract of a mammal and effective to extend the effective life of the drug in the respiratory tract by a time equal to at least twice the effective life of the drug alone.

2. (canceled)

3. The carrier of claim 1 wherein the carrier comprises phosphatidylchol ine.

4. The carrier of claim 3 wherein the carrier further comprises phosphatidylglycerol.

5. The carrier of claim 1 wherein the drug comprises budesonide.

6. The carrier of claim 1 wherein the drug comprises triamcinolone.

7. The carrier of claim 3 wherein the carrier further comprises poly(ethylene glycol).

8. (canceled)

9. (canceled)

10. (canceled)

11. (canceled)

12. The carrier of claim 1 wherein the carrier comprises at least one of poly(ethylene glycol)-conjugated lipids, phosphatidylinositol, dipaimitoylphosphatidylpolyglycerol, lipid conjugated polyoxyethylene, lipid conjugated polysorbate, or lipids conjugated other hydrophilic steric coating molecules safe for in vivo use, the sterically stabilized liposome being effective to extend the effective lifetime of a drug in the respiratory tract of a mammal.

13. (canceled)

14. The carrier of claim 1 wherein the drug is a drug useful for treatment of the respiratory tract of the mammal and is compatible with the sterically stabilized liposome.

15. The carrier of claim 14 wherein the drug is selected from the group consisting of budesonide, flunisolide, triamcinolone, beclomethasone, fluticasone, mometasone, dexamethasone, hydrocortisone, methylprednisolone, prednisone, cotisone, betamethasone, terbutaline, albuterol, ipratropium, pirbuterol, epinephrine, salmeterol, levalbuterol, formoterol, montelukast, zafirlukast, zileuton, loratadine, cetirizine isoniazid, ethainbutol, pyrazinamide, rifamycin; rifampin, streptomycin, clarithromycin, azelastine, theophylline, amikacin, gentamicin, tobramicin, rifabutin, rifapentine, sparfloxacin, ciprofloxacin, quinolones, azithromycin, erythromycin, and isoniazid.

16. The carrier of claim 1 wherein the carrier comprises egg-derived or soybean-derived phosphatidylcholine.

17. The carrier of claim 1 wherein the carrier comprises egg-derived or soybean derived phosphatidylglycerol.

18. A composition comprising a sterically stabilized liposome carrier in combination with a drug, the composition being compatible with a respiratory tract of a mammal, aerosol administration and effective to extend the effective life of the drug in the respiratory tract by a time equal to at least twice the effective life of the drug alone.

19. (canceled)

20. (canceled)

21. (canceled)

22. (canceled)

23. (canceled)

24. (canceled)

25. (canceled)

26. (canceled)

27. (canceled)

29. (canceled)

30. (canceled)

31. (canceled)

32. (canceled)

33. (canceled)

34. (canceled)

35. A method for treating a respiratory tract of a mammal by aerosol administration of an effective amount of a composition comprising a sterically stabilized liposome carrier for combination with a drug, and a drug, the sterically stabilized liposome being compatible with a respiratory tract of a mammal and effective to extend the effective life of the drug in the respiratory tract by a time equal to at least twice the effective life of the drug alone.

36. The method of claim 35 wherein the carrier comprises phosphatidylchol ine.

37. The method of claim 36 wherein the carrier further comprises phosphatidylglycerol.

38. The method of claim 36 wherein the phosphatidylcholine is present in an amount equal to from about 50 to about 100 weight percent.

39. The carrier of claim 37 wherein the carrier comprises from about 0 to about 50 weight percent phosphatidylglycerol.

40. The method of claim 36 wherein the carrier further comprises poly(ethylene glycol).

41. (canceled)

42. The method of claim 35 wherein at least one of phosphatidyicholine, phosphatidylglycerol, and poly(ethylene glycol) attached to a lipid such as phosphatidylethanolamine, have acyl chains containing from about 16 to about 18 carbon atoms.

43. The method of claim 42 wherein the acyl chains contain from about 8 to about 18 carbon atoms.

44. (canceled)

45. The method of claim 35 wherein the carrier comprises at least one of poly(ethylene glycol)-conj ugated lipids, phosphatidylinositol, dipaim itoylphosphatidylpolyglycerol, lipid conjugated polyoxyethylene, lipid conjugated polysorbate, or lipids conjugated other hydrophilic steric coating molecules safe for in vivo use, the sterically stabilized liposome being effective to extend the effective lifetime of a drug in the respiratory tract of a mammal.

46. The method of claim 35 wherein the carrier is phosphatidylcholine, phosphatidylglycerol, poly(ethylene glycol)-distearyolphosphatidyidiethanolamine, with or without cholesterol.

47. The method of claim 35 wherein the drug is a. drug useful for treatment of the respiratory tract of the mammal and is compatible with the sterically stabilized liposome.

48. The method of claim 47 wherein the drug is selected from the group consisting of of budesonide, flunisolide, triamcinolone, beclomethasone, fluticasone, mometasone, dexamethasone, hydrocortisone, methylprednisolone, prednisone, cotisone, betamethasone, terbutaline, albuterol, ipratropium, pirbuterol, epinephrine, salmeterol, levalbuterol, formoterol, montelukast, zafirlukast, zileuton, loratadine, cetirizine isoniazid, ethambutol, pyrazinamide, rifamycin; rifampin, streptomycin, clarithromycin, azelastine, theophylline, amikacin, gentamicin, tobramicin, rifabutin, rifapentine, sparfloxacin, ciprofloxacin, quinolones, azithromycin, erythromycin, and isoniazid.

49. The method of claim 35 wherein the carrier comprises egg-derived or soybean derived phosphatidylglycerol.

50. The method of claim 35 wherein the carrier comprises egg-derived or soybean derived phosphatidylglycerol.

51. The method of claim 35 wherein the drug is budesonide.

52. The method of claim 35 wherein the drug is triamcinolone.
